(19) United States
(12) Patent Application Publication
(10) Pub. No.: US 2013/0298318 A1
(43) Pub. Date: Nov. 14, 2013

(54) Eye Protection for Equestrian Use

(71) Applicant: Kimberly M. Rogers, Somerset, KY (US)

(72) Inventor: Kimberly M. Rogers, Somerset, KY (US)

(73) Assignee: Kimberly Rogers, Somerset, KY (US)

(21) Appl. No.: 13/467,451

(22) Filed: May 9, 2012

(51) Int. Cl. A61F 9/02

(52) U.S. Cl. 2/439

(57) ABSTRACT

Eye protection devices are described that can be utilized in conjunction with a riding helmet for equestrian use. The eye protection device can fit snugly and safely on the wearer's head without impacting the desired fit of the riding helmet. In addition, the eye protection device can include pads on the frame so as to maintain a gap between the frame and the wearer for air flow around the lenses of the eye protection device, which can prevent fogging of the lenses and any visual impairment due to such fogging.

### BACKGROUND

The safety of equestrian activities has increased tremendously with the increased use of protective riding helmets. Typically, the riding helmet is relatively close-fitting, with a retention strap system that connects to the helmet at multiple different points. The attachment points for the strap system generally include two attachment points for each of the left and right straps, with the left and right straps snugly fastening under the wearer's chin.

Unfortunately, between the close-fitting helmet itself and the retention strap system, it is difficult if not impossible to wear eye protection in conjunction with the helmet. For instance, if one were to wear a standard pair of glasses and then place the helmet and retention strap system over the glasses, the glasses would be pressed uncomfortably against the wearer's head and the safe fitting of the protective helmet could be sacrificed. Alternatively, attempts to wear glasses on the outside of the straps, but with the temple arms still sitting behind the ears as necessary leads to a poor fit for the glasses, with the result of the glasses often falling off. Closely fitting goggles, on the other hand, will fog over during use and lead to poor vision.

What is needed in the art is eye protection that can be worn in conjunction with an equestrian helmet that does not interfere with vision of the rider or safety of the helmet and helmet retention system.

### SUMMARY

According to one embodiment, disclosed is an eye protection device for equestrian use. For example, the eye protection device can include a lens, a frame, a pliable band, and a pad. The frame can at least partially surround the lens. The pliable band can be attached to the frame at one end. The pad can be attached to the inner surface of the frame such that during use the pad can maintain a gap between the frame and the wearer of the eye protection device.

In another embodiment, disclosed is an eye protection device for equestrian use that includes a first and second lens, a frame, a right and left pliable band, and a series of pads. The frame can at least partially surround the lenses. The right pliable band can be attached to the right side of the frame at a first end and can include an attachment at the second end. The left pliable band can be attached to the left side of the frame at a first end, and can include an attachment at the second end. The two attachments of the right and left bands can be removably attachable to one another. The series of pads can be attached to the left inner surface and the right inner surface of the frame. During use, the series of pads can maintain a gap between the frame and the wearer of the eye protection device.

### BRIEF DESCRIPTION OF THE FIGURES

A full and enabling disclosure of the present subject matter, including the best mode thereof to one of ordinary skill in the art, is set forth more particularly in the remainder of the specification, including reference to the accompanying figures in which:

FIG. 1 illustrates one embodiment of an eye protection device for equestrian use as described herein.

FIG. 2 is illustrates a left band (FIG. 2A) and a right band (FIG. 2B) as may be utilized in conjunction with the eye protection device as disclosed herein.

FIG. 3 is a bottom view of one embodiment of the eye protection device.

FIG. 4A provides a close view of an attachment point between the right band and the frame of an eye protection device.

FIG. 4B is a top view of one embodiment of the eye protection device.

FIG. 5 is a front view of one embodiment of the eye protection device.

FIG. 6 is a back view of one embodiment of the eye protection device.

FIG. 7 illustrates the eye protection during use in conjunction with a riding helmet.

FIG. 8 illustrates the eye protection during use in conjunction with a riding helmet.

FIG. 9 illustrates the fit of the eye protection during use.

FIG. 10 illustrates a method for removing the eye protection while wearing a riding helmet.

FIG. 11 illustrates the removal of the eye protection during use while wearing a riding helmet.

Repeat use of reference characters in the present specification and drawings is intended to represent the same or analogous features or elements of the present invention.

### DETAILED DESCRIPTION

Reference will now be made in detail to various embodiments of the disclosed subject matter, one or more examples of which are set forth below. Each embodiment is provided by way of explanation of the subject matter, not limitation thereof. In fact, it will be apparent to those skilled in the art that various modifications and variations may be made in the present disclosure without departing from the scope or spirit of the subject matter. For instance, features illustrated or described as part of one embodiment, may be used in another embodiment to yield a still further embodiment.

In general, the present disclosure is directed to eye protection devices that can be utilized in conjunction with a riding helmet for equestrian use. The eye protection device can fit snugly and safely on the wearer's head without impacting the desired fit of the riding helmet. In addition, the eye protection device can include pads so as to maintain a gap between the frame and the wearer for air flow around the lenses of the eye protection device, which can prevent fogging of the lenses and any visual impairment due to such fogging.

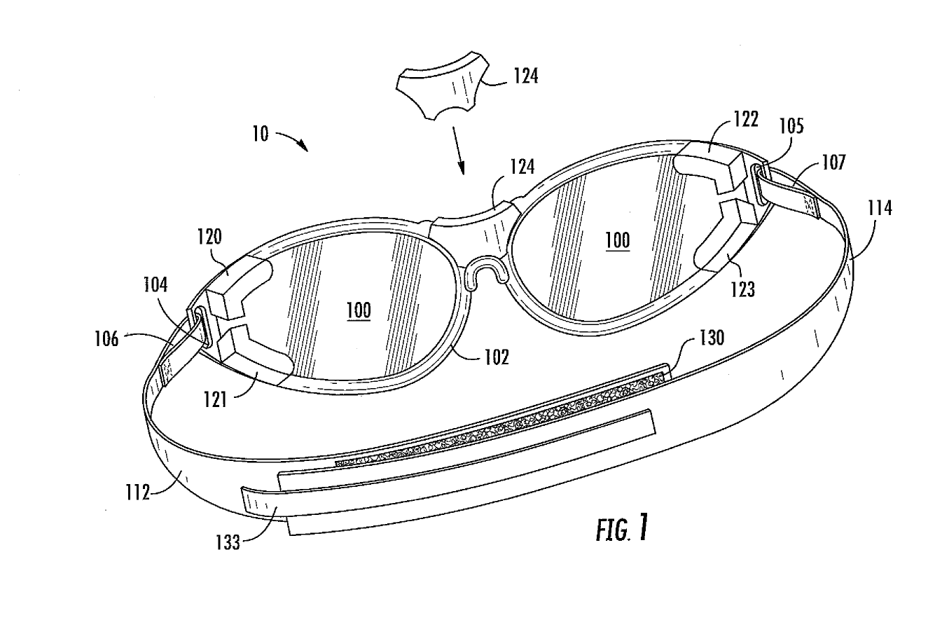

FIG. 1 illustrates one embodiment of the eye protection device 10. As can be seen, the eye protection device includes lenses 100, left and right bands 112, 114, and pads 120, 121, 122, 123, 124. The lenses 100 can generally be formed of eye lens materials including shatter proof polymers, glasses, and so forth, as well as laminated materials. By way of example, lenses 100 can be formed of a shatter-proof material such as a polycarbonate or an acrylic as is generally known in the art. Though illustrated in FIG. 1 as including two separate lenses 100, the eye protection device can include a single lens that extends across the device to allow clear vision through the lens.

The lenses 100 can be corrective lenses or can be provided merely for protection. For instance, the lenses 100 can be designed to protect the wearer from potential hazards including solid hazards such as dirt, leaves, insects, etc. that may be encountered while riding. In this embodiment, the lenses can conform to standards required for classification as safety glasses, for instance the lenses can conform to standard ANSI Z87.1-2003 for basic impact lenses or alternatively for high impact lenses. In accord with the standard, basic impact lenses will pass the “drop ball” test, in which a 1 inch diameter steel ball is dropped on the lens from 50 inches high. High impact lenses will pass the “high velocity” test in which 0.25 steel balls are shot at the lenses from various velocities. The ANSI Z87.1-2003 standard is available from the American National Standards Institute, as is known.

Alternatively or in addition to providing vision correction and/or protecting the wearer from solid hazards, the lenses can protect the wearer from sunlight. Thus, the lenses may be tinted or photochromic so as to function as sunglasses. The function of the lenses for sun protection can be in addition to or alternative to functioning as protective safety glasses and/or corrective lenses.

The lenses 100 can be encloses by a frame 102 that can be formed of a molded shatter proof material such as a polymeric material or a metal. The frame can have any desired shape. For instance, though illustrated in FIG. 1 as completely enclosing the lenses 100, in other embodiments, the frame 102 may partially enclose the lenses.

Moreover, it should be understood that the frame 102 need not be formed of a separate material that is attached to the lens 100. For example, in one embodiment the eye protection device may include a unitary lens and frame construction in which the lens and the frame are formed of the same, continuous material. In this embodiment, the frame may attach to other components of the eye protection device as described but is of a continuous, unitary construction with the lens, rather than a separate material.

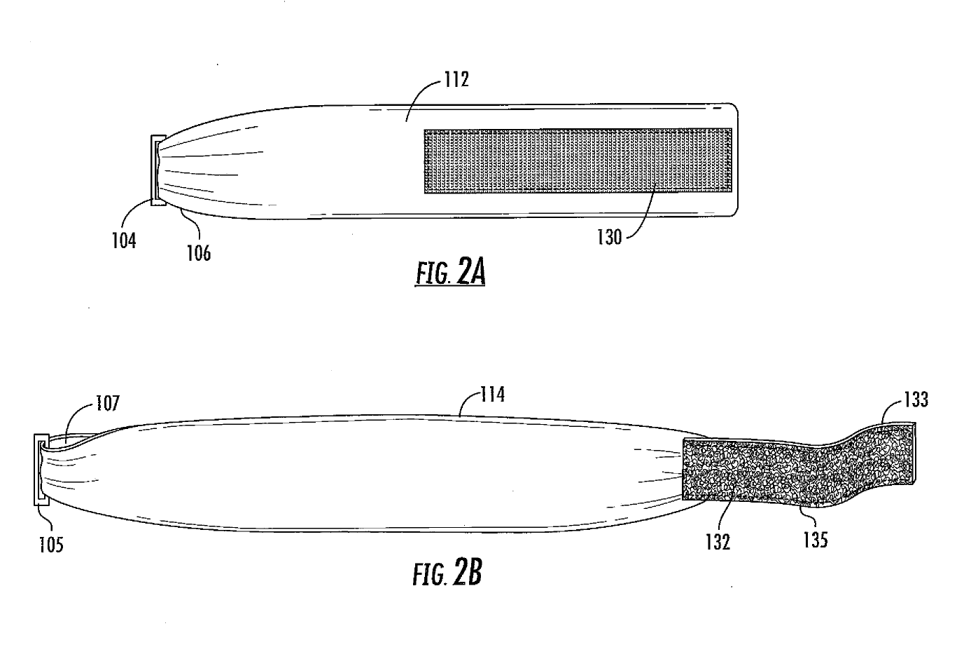

In the embodiment illustrated in FIG. 1, the left band 112 and right band 114 are attached to either side of the frame 102. As utilized herein, the terms ‘left’ and ‘right’ are used from the perspective of a wearer of the eye protection device. FIG. 2 provides additional views of left band 112 (FIG. 2A) and right band 114 (FIG. 2B). The left band 112 and the right band 114 can be formed of a pliable material so as to fit snugly against the wearer's head. For example, the left band 112 and the right band 114 can be formed of the same or different woven or nonwoven fabrics. In one embodiment, fabrics for use in forming the left and right bands 112, 114 can be absorbent, for instance an absorbent nonwoven fleece, that can absorb moisture during use and prevent visual impairment due to moisture getting into the eyes of the wearer. In one embodiment, fabrics useful for forming the left and right bands 112, 114 can exhibit elasticity, which can be useful to maintain a snug fit of the eye protection device during use.

The left and right bands 112, 114 can include attachments for attaching the bands to the frame 102. For instance, in the embodiment of FIG. 2, the left band 112 includes a loop 106 at a first end that can be used to attach the left band 112 to the frame 102. Similarly, the right band 114 includes a loop 107 at a first end that can be used to attach the right band 114 to the frame 102. The loops 106, 107 can be formed by doubling the first end of the band over itself and adhering the end of the band to the body of the band by sewing, use of an adhesive, or the like. Alternatively, the loops 106, 107 can be formed by attaching a separately formed loop of the same or a different material as used to form the main body of the bands on to the end of each band 112, 114.

The size of the left and right bands 112, 114 can vary as desired to provide a comfortable, safe fit to the wearer. The width of the bands can be designed so as to remain in place on the wearer's head without feeling excessively tight and without covering so much of the wearer's head so as to interfere with fit or comfort of the helmet. In general, the bands 112, 114 can be between about 0.25 inches and about 3 inches in width.

The bands 112, 114 can be of a length so as to meet at the back of the wearer's head during use and, depending upon the attachment mechanism used, to optionally overlap during use. For example, the bands 112, 114 can generally be between about 3 inches and about 10 inches in length. In addition the bands 112, 114 can be the same length as one another or of different lengths, as shown.

The bands 112, 114 can include attachments 130, 132 for temporarily securing the second ends of the bands 112, 114 to one another during use. For example, in the embodiment illustrated in FIGS. 1 and 2 the attachments 130, 132 can be the opposing sides of a hook and loop style attachment, such as a Velcro® tape. Of course, other attachments can alternatively be utilized such as snaps, interlocking buckles, strap/buckle connections, buttons, ties, and so forth. Moreover, though illustrated with the right band 114 overlaying the left band 112 in FIG. 1, either band may alternatively overlay the other upon attachment or, alternatively, the two ends of the bands 112, 114 may meet upon attachment.

Upon attachment to one another, the bands 112, 114 can form a single band that passes from a first attachment point of the frame 102 to a second attachment point of the frame 102 and around the wearer's head. When attached to one another around a wearer's head, the bands can pass over the ears, so as to not impede the wearer's hearing. The bands can be located around the wearer's head so as to provide a secure fit, which can be selected for each wearer individually. In general, however, the bands can remain beneath the base of the helmet during use. As such, the eye protection device can be easily removed without the necessity of first removing the helmet.

In another embodiment (not shown), a single band can be utilized rather than two separate bands. In this embodiment, the single band can extend from a first attachment point of the frame 102 to a second attachment point of the frame. The single band can be removably attachable to the first and/or the second attachment points of the frame.

In yet another embodiment, the single band can be permanently attached to either side of the frame and the single band can be elastic so as to be stretched over the wearer's head to don and remove the glasses. However, this particular embodiment would necessitate the removal of the rider's helmet in order to don and remove the eye protection device, and thus may not be preferred in some situations.

The attachments used to connect the bands 112, 114 to one another (or to connect a single band to the frame) can also include a quick release mechanism for ease in removing the eye protection device. For example, in the illustrated embodiment, in which the left and right bands 112, 114 overlap one another upon attachment, the band that will be external to the wearer's head during use, in this case the right band 114, can include a tab 113 that can be pulled to separate the left and right band 112, 114 from one another to remove the eye protection device following use. The tab 133 can be merely an extension of the right band 114 that extends beyond the attachment 132 or can be a portion of a separate piece 135 that has been sewn or otherwise attached to the second end of the right band 114. As can be seen with reference to FIG. 2, the attachment 132 can also be located on the separate piece 135 that has been attached to the second end of the right band 114. Of course, this is not a requirement of the protection eye device, and the attachment 132 can be located directly on the right band 114. Moreover, the tab 113 can be located on the left band 112, rather than the right band 114. In addition, both the left and right bands 112, 114 can include a tab, so as to allow for facile separation of the bands 112, 114 from one another by the wearer using either the right hand or the left hand to pull the tab 113 and separation the bands 112, 114 from one another.

Referring again to FIG. 1, the left and right bands 112, 114 are attached to the frame 102 at left and right slots 104, 105, respectively. Left and right slots 104, 105 are formed into the frame 102 as shown. Slots 104, 105 can be closed slots a shown or alternatively can be open at the top, which would allow for removable attachment of the bands 112, 114 to the frame 102 and the simple removal of the left and right bands 112, 114 from the frame 102 by merely sliding the left and right bands 112, 114 over the top of the slots 104, 105. In general, however, the slots 104, 105 can be closed, so as to prevent undesired detachment of the left band 112 or the right band 114 from the frame 102.

In those embodiments in which the slots 104, 105 are closed, the respective ends of the left and right bands 112, 114 can be passed through the slots and then attached to the body of the bands 112, 114 forming loops 106, 107 at the same time as attaching the bands 112, 114 to the frame 102. Of course, any other mechanism for attachment of the bands 112, 114 can alternatively be utilized, such as use of an adhesive, thermal bonding, etc.

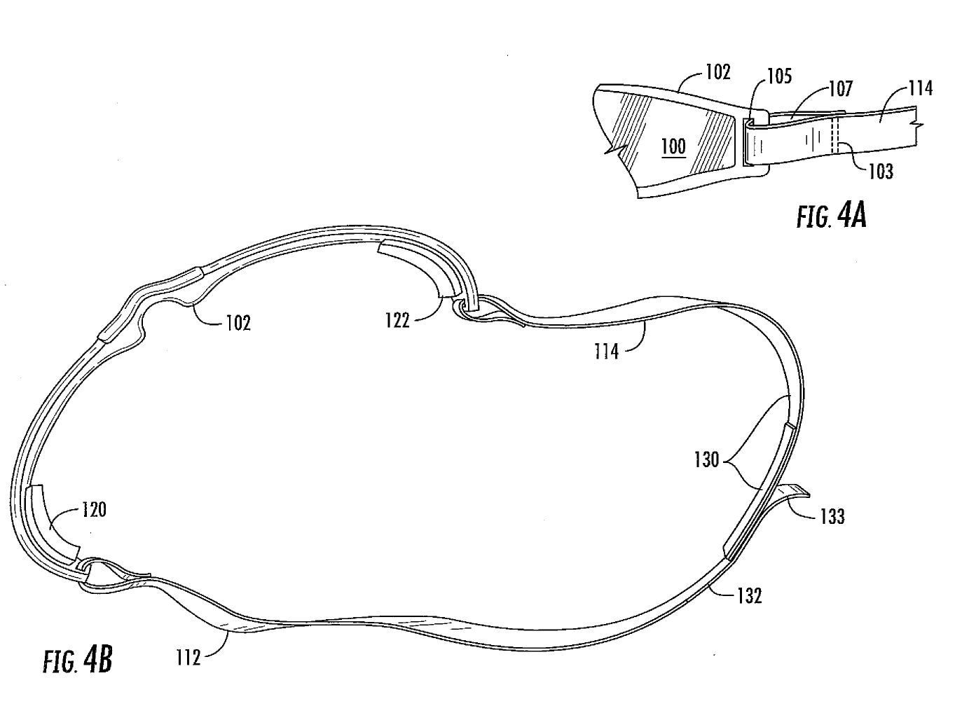

FIG. 4A presents a closer view of the attachment of right band 114 to the frame 102. As can be seen, the end of right band 114 is passed through the closed slot 105 that is formed into the frame 102. The end of the right band 114 is then attached to the body of the right band 114 at seam 103 to form loop 107 and to securely attach the right band 104 to the frame 102.

The eye protection device 10 also includes a series of pads 120, 121, 122, 123 that are located on the inner surface of the frame 102. As utilized herein, the term ‘inner’ generally refers to a surface that is facing toward the wearer during use of the device. Specifically, pads 120, 121 are located on the inner left side of the frame 102 and pads 122, 123 are located on the inner right side of the frame. During use, the pads 120, 121, 122, 123 can rest against the side of the wearer's head, generally in the temple region, and can ensure a gap is maintained between the wearer's head and the frame 102. The gap between the wearer's head and the frame 102 will allow air flow behind the lenses of the device 10, which can prevent fogging of the lenses 100 during use. In addition, the pads 120, 121, 122, 123 can prevent the frame 102 from uncomfortably pressing against the wearer's head upon attachment of the bands 112, 114 to one another, and thus can improve the comfort of the device 10 during use.

In general, the pads 120, 121, 122, 123 can be formed of a relatively soft material, such as a foam, a gel, or an elastomeric polymer in order that the bands 112, 114 can be attached to one another and securely wrap behind the wearer's head without the necessity of the pads 120, 121, 122, 123 pressing uncomfortably against the wearer's head. By way of example, the pads 120, 121, 122, 123 can be formed of a polyurethane, silicone, or thermoplastic rubber, or a viscoelastic gel material. For example, the pads can be formed of materials including, and without limitation to, styrene-olefin-rubber block copolymers, thermoplastic polyurethanes, thermoplastic polyolefins, polyamides, polyureas, and polyesters, thermoplastic elastomers, polystyrenes, polyisoprenes, natural and synthetic rubbers, silicon rubbers, nitrile rubbers, butyl rubbers, foamed polymers, etc.

The pads 120, 121, 122, 123 can be adhered to the frame 102 by use of a standard adhesive, for instance a pressure sensitive adhesive such that the pads 120, 121, 122, 123 can be located at the desired locations, for instance by the wearer following purchase of the eye protection device, and then adhered to the frame 102. Any suitable adhesive may be utilized including, without limitation, an epoxy adhesive, a silicone adhesive, a urethane adhesive, or the like. As stated, in one embodiment, the pads 120, 121, 122, 123 can be provided to the wearer unattached to the frame 102 in order that the wearer can locate the pads as desired to provide an individualized fit for the device 10. Alternatively, the pads 120, 121, 122, 123 can be attached to the frame 102 during formation of the device 10 by the manufacturer.

The eye protection device 10 also includes a nose pad 124 that can be formed of the same material or a different material as the pads 120, 121, 122, 123. Nose pad 124 can be located over the nose bridge of the frame 102 and can provide additional comfort to the wearer as well as providing additional air flow between the wearer and the lenses 100. The utilization of nose pad 124 in conjunction with the other pads 120, 121, 122, 123 is not required, however, and in other embodiments, the eye protection device can include pads 120, 121, 122, 123 without the addition of nose pad 124.

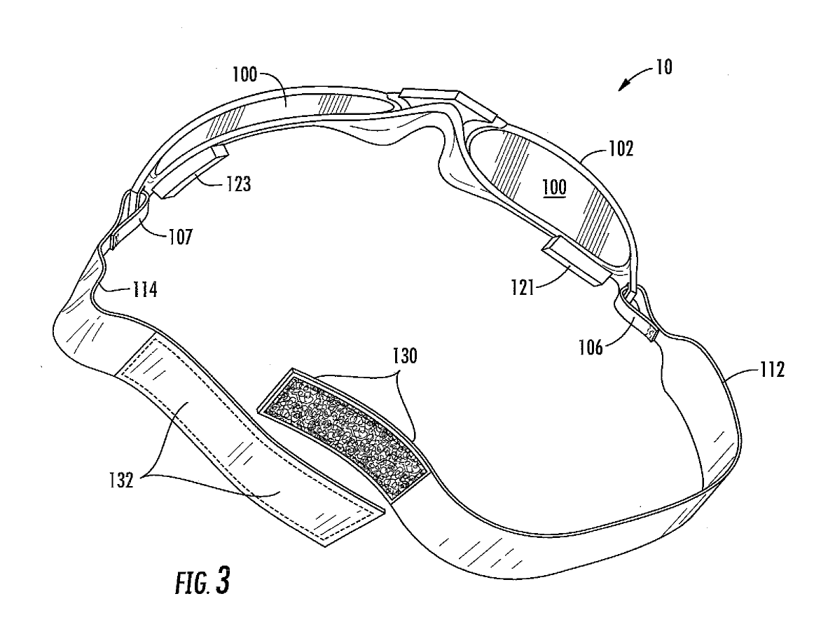

FIG. 3 presents a bottom view of an eye protection device 10. As can be seen, the device includes left band 112 and right band 114 with attachments 130, 132 on each band, respectively. The attachments 130, 132 are provided so as to overlap, as shown, which can allow for the attachment between the bands 112, 114 to be adjustable and provide an individualized fit for each wearer. Of course, other adjustable attachment mechanisms as are generally known in the art can alternatively be utilized, including straps and buckles, and so forth.

Also visible in FIG. 3 is the base of the frame 102, which can include an amount of curvature so as to provide a better fit to the wearer. As can be seen, the frame 102 can extend laterally for a distance beyond the outer edge of the pads 121, 123, which can provide peripheral protection to the wearer as well as frontal protection. Of course, the extension of the frame 102 to either side of the device can vary so as to provide variable levels of side protection as well as a variety of aesthetically pleasing frame shapes.

FIG. 4B presents a top view of the eye protection device 10. In this view, the left band 112 and the right band 114 are attached to one another by use of attachments 130, 132. As can be seen, the right band 114 also includes tab 133. In order to remove the device 10, a wearer can simply pull the tab 133 and detach the left and right bands 112, 114 from one another. Moreover, the left and right bands 112, 114 can be detached from one another without the need of first removing the wearer's riding helmet. Ease of removal can be particularly beneficial when considering the utilization of the eye protection device 10 for children.

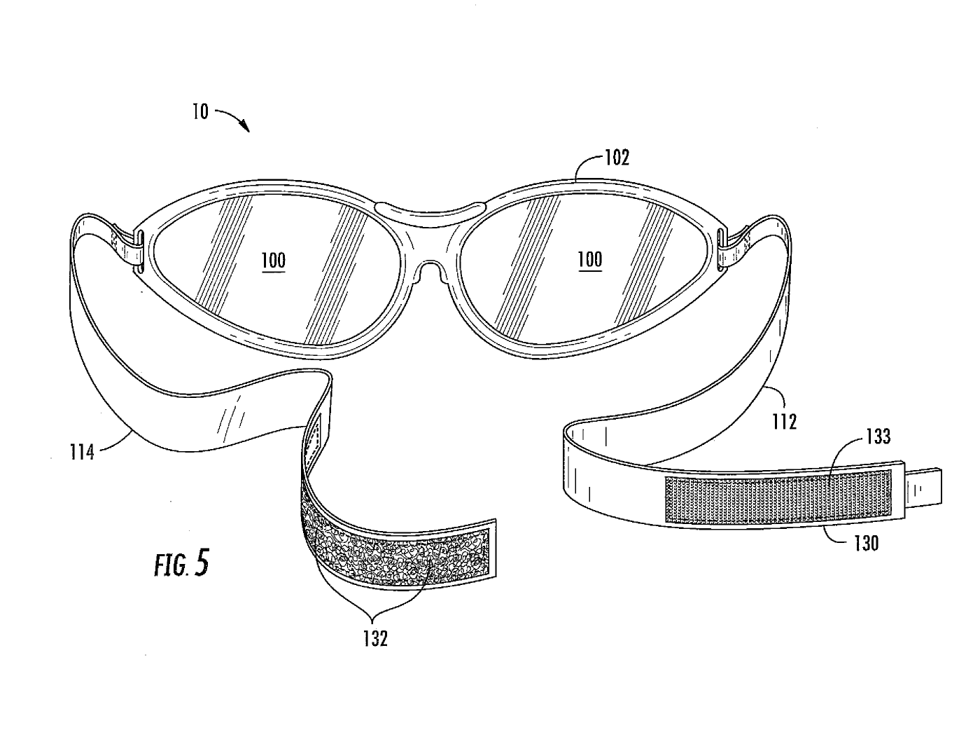

A front view of the eye protection device 10 is provided in FIG. 5. As can be seen, the pads that provide for both comfort and improved function of the device are not visible in the front view. This can improve the aesthetics of the device during use. In addition, the shape of the frame 102 and lenses 100 can be varied as desired to provide any desired ‘look’ to the device 10 and an aesthetically pleasing device to any wearer. For example, in addition to a variety of tints, colors, shapes, etc. of the lenses 100, as previously discussed, the frame 102 can be shaped and/or colored as desired. In addition the left band 112 and right band 114 can be of any color, pattern, etc. to provide a widely variable look to the device 10.

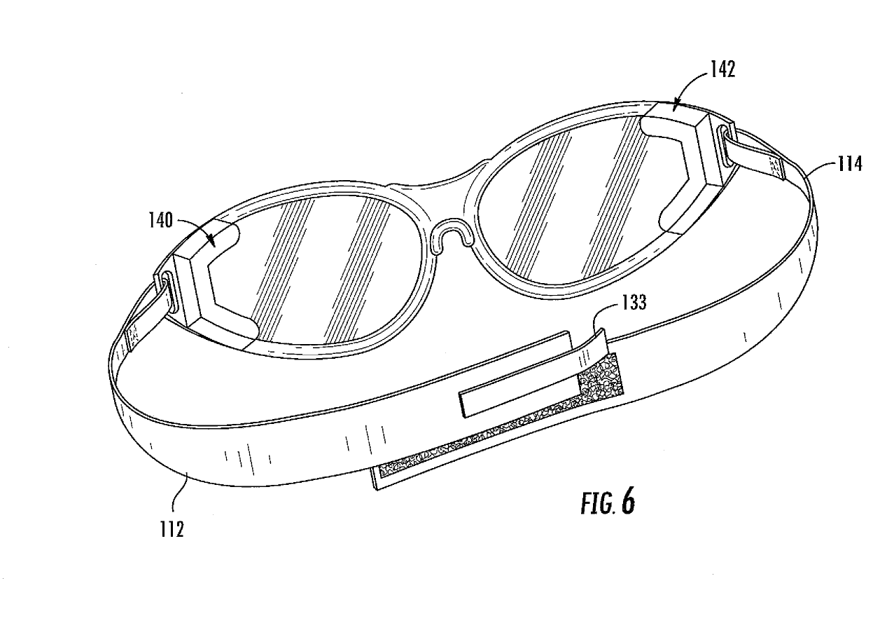

FIG. 6 presents another embodiment of an eye protection device 20. As can be seen, in this embodiment, a single pad 140 is located at the left side of the frame 102 and a single pad 142 is located at the right side of the frame 102, which differs from the multiple pads located at either side of the frame in the embodiment of FIG. 1. Single pads 140, 142 can be shaped to rest comfortably against the wearer's head in the temple region and maintain a gap between the frame and the wearer's head during use. Of course, the number of pads on either side of the frame is not limited to one or two and any number of pads can be located on the frame so as to maintain the desired gap as well as a safe and comfortable fit for the eye protection device. Eye protection device 20 also does not include any nose pad, which is an optional element as discussed previously.

During use, the eye protection device can be worn with a riding helmet without affecting the proper fit or comfort of the riding helmet. FIG. 7 illustrates use of a device 10 in conjunction with use of a riding helmet 200. As can be seen, the left band 112 passes over the ear and under the retaining straps 202, 204 of the helmet 200 without interference with the helmet. In addition, as the left band 112 passes over the ear of the wearer, the eye protection device 10 will not interfere with the wearer's hearing.

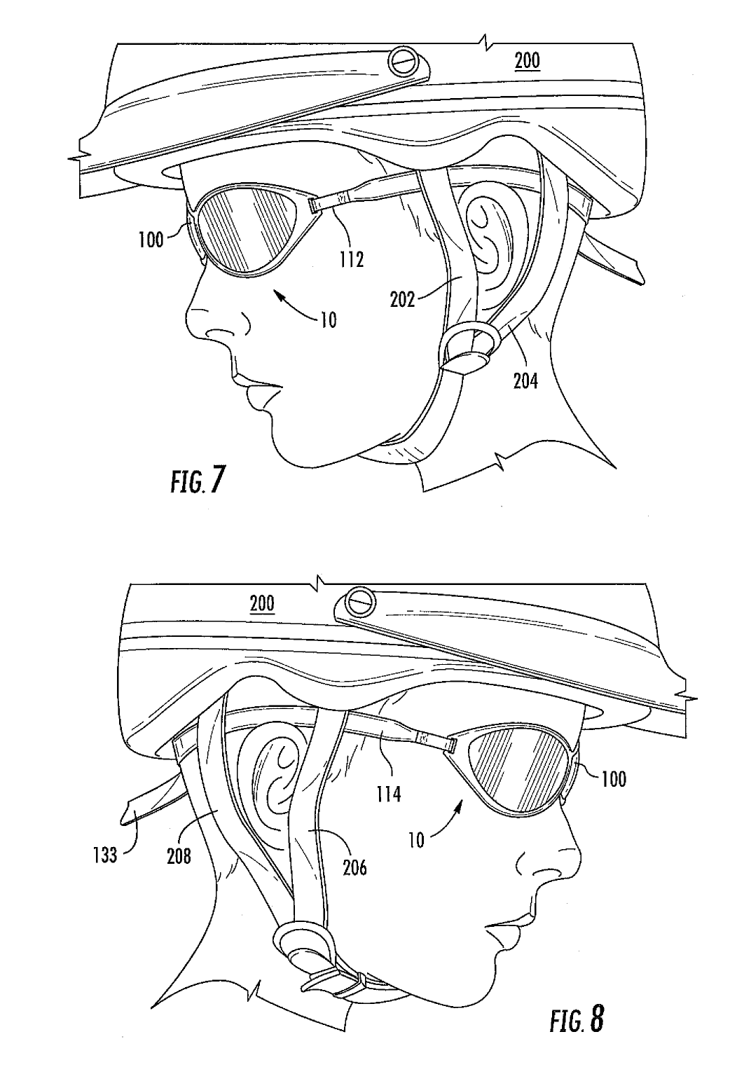

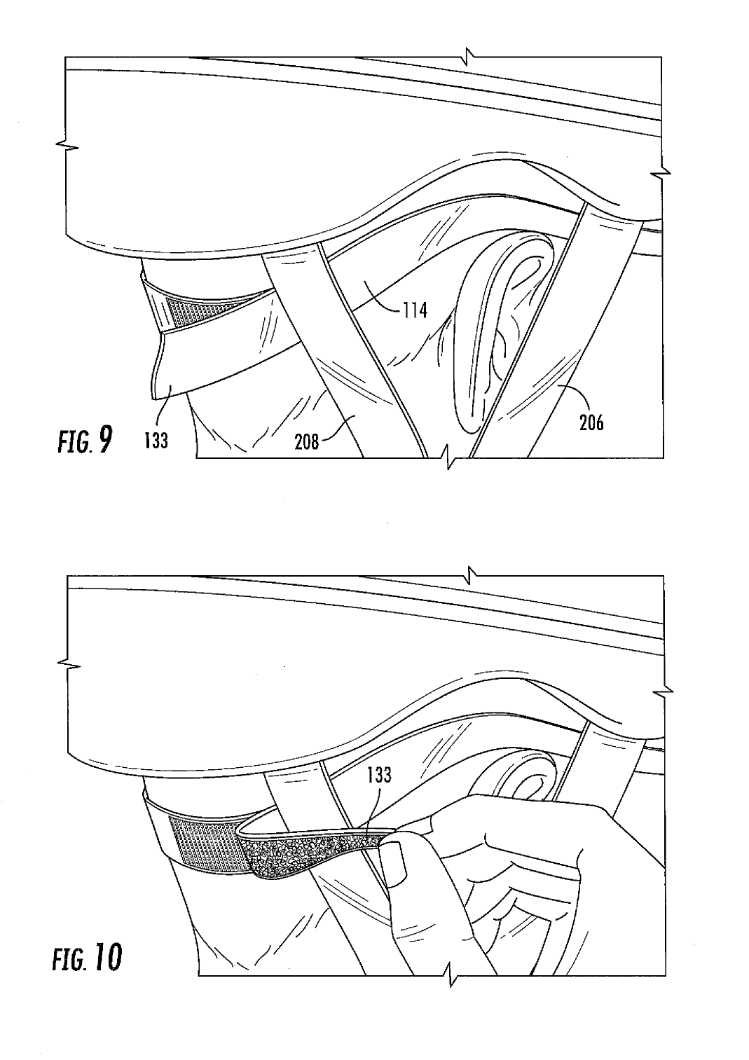

FIG. 8 illustrates the device 10 during use from the opposite side of the wearer. In this view can be seen the right band 114 passing over the ear and under the helmet retaining straps 206, 208 on the right side of the wearer's head. In addition, in this view can be seen the tab 133 at the back of the eye protection device that can be used to remove the eye protection device without the necessity of also removing the helmet 200. FIG. 9 illustrates a closer view of the tab 133 that associated with right band 114. During use, tab 133 is to the rear of the retaining straps 206, 208 so as to be accessible to the wearer.

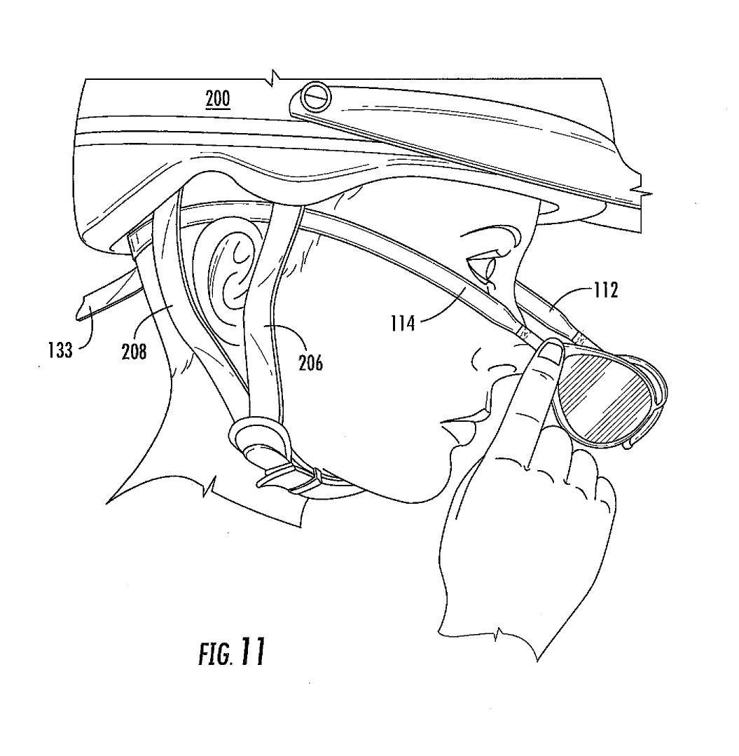

To remove the eye protection device 10 without also removing the helmet 200, the wearer can simply pull the tab 133 so as to disassociate the left and right bands from one another, as shown in FIG. 10. For example, when considering a pull apart-type of attachment for connection of the bands 112, 114 to one another, such as a Velcro® tape, one or more snaps, or the like, the wearer can pull on the tab 133 to disconnect the bands 112, 114 from one another and remove the eye protection device from beneath the helmet 200. FIG. 11 illustrates such removal in which the left and right bands 112, 114 can simply slide out from under the retaining straps as shown without the necessity of also removing the helmet 200.

While certain embodiments of the disclosed subject matter have been described using specific terms, such description is for illustrative purposes only, and it is to be understood that changes and variations may be made without departing from the spirit or scope of the subject matter.

What is claimed is:

1. An eye protection device for equestrian use comprising:
a lens;
a frame at least partially surrounding the lens, the frame including an inner surface;
a pliable band comprising a first end and a second end, the pliable band being attached to the frame at the first end of the pliable band; and
a pad attached to the inner surface of the frame, wherein during use, the pad maintains a gap between the frame and the wearer of the eye protection device for equestrian use.

2. The eye protection device according to claim 1, wherein the lens is a basic impact lens.

3. The eye protection device according to claim 1, wherein the lens is a high impact lens.

4. The eye protection device according to claim 1, wherein the lens is a tinted or a photochromic lens.

5. The eye protection device according to claim 1, wherein the lens is a corrective lens.

6. The eye protection device according to claim 1, wherein the lens and the frame are of unitary construction.

7. The eye protection device according to claim 1, wherein the pliable band is a fabric band.

8. The eye protection device according to claim 1, the pliable band further comprising an attachment at the second end of the pliable band.

9. The eye protection device according to claim 1, further comprising a quick release mechanism at the second end of the pliable band.

10. The eye protection device according to claim 1, wherein the pliable band is removably attachable to the frame.

11. The eye protection device according to claim 1, wherein the pad comprises a foam, a gel, or an elastomeric polymer.

12. An eye protection device for equestrian use comprising:
a first lens;
a second lens;
a frame at least partially surrounding the first lens and the second lens, the frame defining a left side, a right side, left inner surface, a right inner surface, and a nose bridge;
a right pliable band, the right pliable band comprising a first end and a second end, the first end of the right pliable band being attached to the right side of the frame, the second end of the right pliable band comprising a first attachment;
a left pliable band, the left pliable band comprising a first end and a second end, the first end of the left pliable band being attached to the left side of the frame, the second end of the left pliable band comprising a second attachment that is removably attachable to the first attachment; and
a series of pads attached to the left inner surface and the right inner surface of the frame, wherein during use, the series of pads maintain a gap between the frame and the wearer of the eye protection device for equestrian use.

13. The eye protection device according to claim 12, wherein the first and second lenses are basic impact lenses.

14. The eye protection device according to claim 12, wherein the first and second lenses are high impact lenses.

15. The eye protection device according to claim 12, wherein the first and second lenses are tinted or photochromic lenses.

16. The eye protection device according to claim 12, wherein the first and second lenses are corrective lenses.

17. The eye protection device according to claim 12, wherein the right and left pliable bands are fabric bands.

18. The eye protection device according to claim 12, further comprising a nose pad attached to the nose bridge of the frame.
